(19) United States
(12) Patent Application Publication
(10) Pub. No.: US 2013/0218003 A1
(43) Pub. Date: Aug. 22, 2013

(54) RAPID ENTRY POINT LOCALIZATION FOR PERCUTANEOUS INTERVENTIONS

(71) Applicants: Eva Rothgang, Nurnberg (DE); Wesley David Gilson, Pasadina, MD (US)

(72) Inventors: Eva Rothgang, Nurnberg (DE); Wesley David Gilson, Pasadina, MD (US)

(73) Assignees: Siemens Aktiengesellschaft, Munich (DE); Siemens Corporation, Iselin, NJ (US)

(21) Appl. No.: 13/772,855

(22) Filed: Feb. 21, 2013

Related U.S. Application Data
(60) Provisional application No. 61/601,256, filed on Feb. 21, 2012.

(51) Int. Cl. A61B 5/06; A61B 8/00; A61B 5/055; A61B 6/04; A61B 6/03; A61B 8/08

(52) U.S. Cl. CPC A61B 5/062; A61B 6/03; A61B 8/0841; A61B 8/483; A61B 5/0555; A61B 6/0407; A61B 8/40
USPC 600/414; 600/424

(57) ABSTRACT

A method for localizing a skin entry point on a patient for a percutaneous intervention includes planning a needle trajectory for the percutaneous intervention using a 3D planning image dataset and a planning application, performing a superior-inferior localization of an imaging scanner table containing an imaging scanner using the needle trajectory, and performing a lateral localization of the skin entry point using the needle trajectory.

### CROSS REFERENCE TO RELATED UNITED STATES APPLICATIONS

This application claims priority from “Rapid Physical Identification of a Location on an Object Surface from Tomographic Images”, U.S. Provisional Application No. 61/601,256 of Rothgang, et al., filed Feb. 21, 2012, the contents of which are herein incorporated by reference in their entirety.

### TECHNICAL FIELD

This disclosure is directed to methods for locating a physical entry point for a needle on a patient's skin using digital imaging techniques.

### DISCUSSION OF THE RELATED ART

Recent open and wide bore scanners, combined with the advantages inherent to magnetic resonance (MR) imaging, have led to an increased interest in using MR for guidance of minimally invasive percutaneous interventions like aspiration, biopsy, sclerotherapy, targeted drug delivery and thermal ablation. All of these procedures require the identification of a skin entry site for needle placement. Even though this sounds straightforward, it is often a time-consuming process as the entry site is usually identified in an iterative fashion under real-time imaging using a fingertip or a water-filled syringe.

### SUMMARY

Exemplary embodiments of the invention as described herein generally include methods for rapidly, accurately, and reproducibly localizing a skin entry site without the need for additional imaging or hardware. A method according to an embodiment of the invention, can localize a skin entry site using only the landmark laser built into every MR scanner and image processing methods, eliminates the need for additional entry point localization imaging, reduces the overall procedure time, and can be performed on any clinical scanner.

According to an aspect of the invention, there is provided a method for localizing a skin entry point on a patient for a percutaneous intervention, including planning a needle trajectory for the percutaneous intervention using a 3D planning image dataset and a planning application, performing a superior-inferior localization of an imaging scanner table containing an imaging scanner using the needle trajectory, and performing a lateral localization of the skin entry point using the needle trajectory.

According to a further aspect of the invention, performing a superior-inferior localization of an imaging scanner table comprises translating the imaging scanner table so that a landmark laser of the imaging scanner delineates an axial slice location in the 3D planning image dataset corresponding to the skin entry point.

According to a further aspect of the invention, translating an imaging scanner table is determined from tₘₒᵥₑ=d_{iso,laser}+t_{curr}_{_{—}}ₚₒₛ±e_{z}, wherein d_{iso,laser} is a distance between the landmark laser and an isocenter of the imaging scanner magnet, t_{curr}_{_{—}}ₚₒₛ is a current scanner table position and e_{z} is a z-coordinate of the skin entry point.

According to a further aspect of the invention, the e_{z} term is added if the patient is registered head first with the imaging scanner, and the the e_{z} term is subtracted if the patient is registered feet first with the imaging scanner.

According to a further aspect of the invention, performing a lateral localization of the skin entry point includes segmenting an object in the axial slice corresponding to the skin entry point, generating a curved line along an edge of the segmented object from the skin entry point to a zero x coordinate, and determining a length of the curved line, wherein the curve length defines a lateral offset of the skin entry point.

According to a further aspect of the invention, a landmark laser of the imaging scanner is illuminating the zero x coordinate with cross hairs, and the skin entry point corresponds to a lateral position of the laser light cross hairs.

According to a further aspect of the invention, segmenting an object is performed using a minimum error thresholding technique.

According to another aspect of the invention, there is provided a system for localizing a skin entry point on a patient for a percutaneous intervention, including an imaging scanner disposed on an imaging scanner table, said imaging scanner configured to acquire imaging data from a patient and including a landmark laser, a planning application configured to plan a needle trajectory for the percutaneous intervention using a 3D planning image dataset, a superior-inferior localizer configured to translate the imaging scanner table so that the landmark laser of the imaging scanner delineates an axial slice location in the 3D planning image dataset corresponding to the skin entry point, and a lateral localizer configured to measure a distance along the patient's skin from the skin entry point to a point marked on the patient's skin by the landmark laser.

According to a further aspect of the invention, the imaging scanner table translation is determined from tₘₒᵥₑ=d_{iso,laser}+t_{curr}_{_{—}}ₚₒₛ±e_{z}, wherein d_{iso,laser} is a distance between the landmark laser and an isocenter of the imaging scanner magnet, t_{curr}_{_{—}}ₚₒₛ is a current scanner table position, e_{z} is a z-coordinate of the skin entry point, wherein the e_{z} term is added if the patient is registered head first with the imaging scanner, and the the e_{z} term is subtracted if the patient is registered feet first with the imaging scanner.

According to a further aspect of the invention, the imaging scanner is a magnetic resonance imaging scanner.

According to another aspect of the invention, there is provided a non-transitory program storage device readable by a computer, tangibly embodying a program of instructions executed by the computer to perform the method steps for localizing a skin entry point on a patient for a percutaneous intervention.

### BRIEF DESCRIPTION OF THE DRAWINGS

FIG. 1 illustrates the use of a built-in landmark laser for physical entry point localization, according to an embodiment of the invention.

FIG. 2 illustrates a planning application used to define the trajectory by setting entry and target points in MPR planes, according to an embodiment of the invention.

FIGS. 3(A)-(B) depicts an axial slice corresponding to the skin entry site used for calculating the L-R offset, according to an embodiment of the invention.

FIG. 4 illustrates verification imaging slices aligned along the planned trajectory, according to an embodiment of the invention.

FIG. 5 is a flowchart of a method for localizing a skin entry site, according to an embodiment of the invention.

FIG. 6 is a block diagram of an exemplary computer system for localizing a skin entry site, according to an embodiment of the invention.

### DETAILED DESCRIPTION OF EXEMPLARY EMBODIMENTS

Exemplary embodiments of the invention as described herein generally include systems and methods for localizing a skin entry site. Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention.

As used herein, the term “image” refers to multi-dimensional data composed of discrete image elements (e.g., pixels for 2-dimensional images and voxels for 3-dimensional images). The image may be, for example, a medical image of a subject collected by computer tomography, magnetic resonance imaging, ultrasound, or any other medical imaging system known to one of skill in the art. The image may also be provided from non-medical contexts, such as, for example, remote sensing systems, electron microscopy, etc. Although an image can be thought of as a function from R³ to R or R⁷, the methods of the inventions are not limited to such images, and can be applied to images of any dimension, e.g., a 2-dimensional picture or a 3-dimensional volume. For a 2- or 3-dimensional image, the domain of the image is typically a 2- or 3-dimensional rectangular array, wherein each pixel or voxel can be addressed with reference to a set of 2 or 3 mutually orthogonal axes. The terms “digital” and “digitized” as used herein will refer to images or volumes, as appropriate, in a digital or digitized format acquired via a digital acquisition system or via conversion from an analog image.

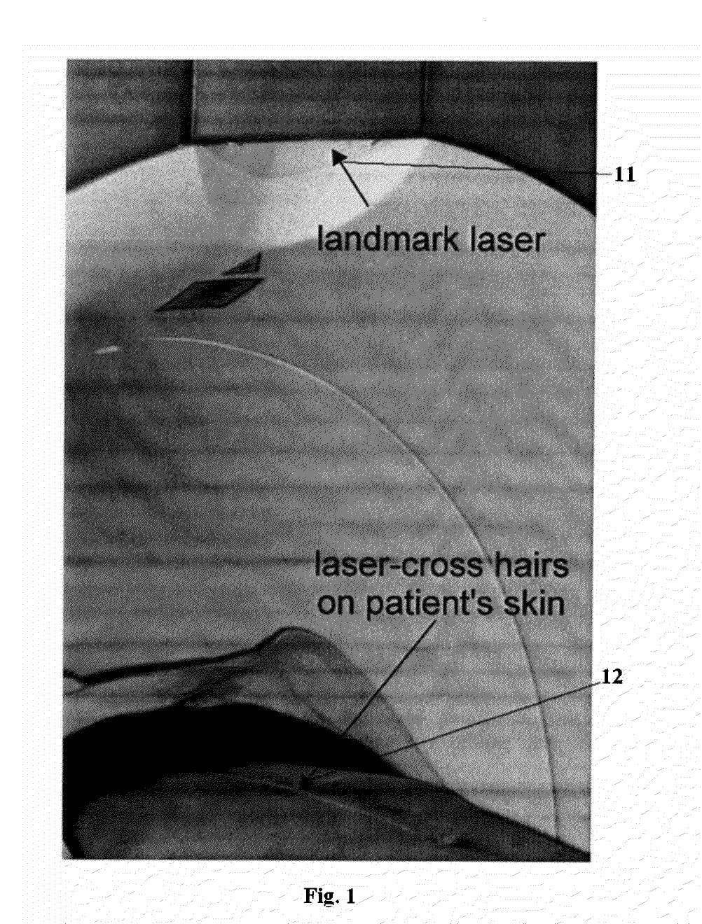

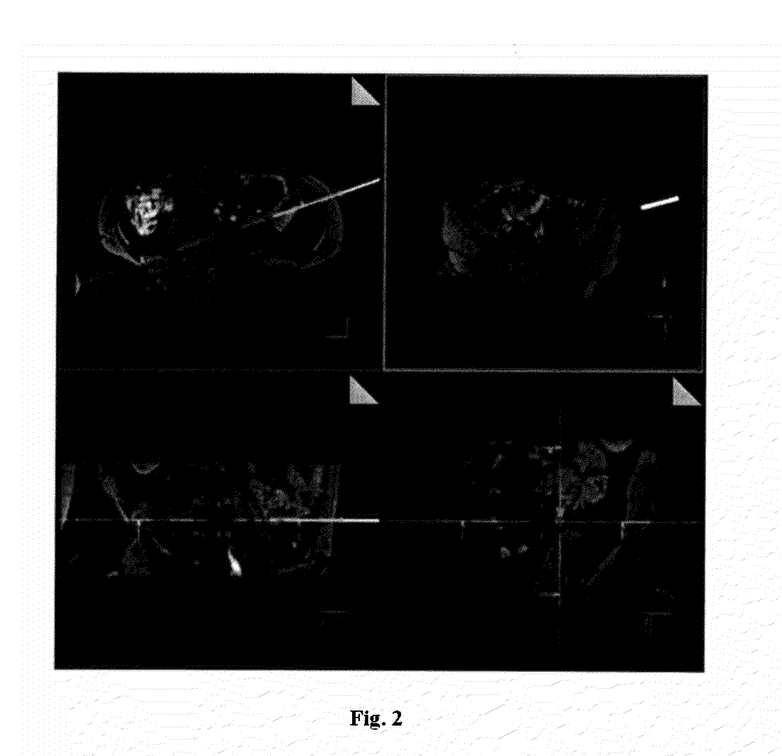

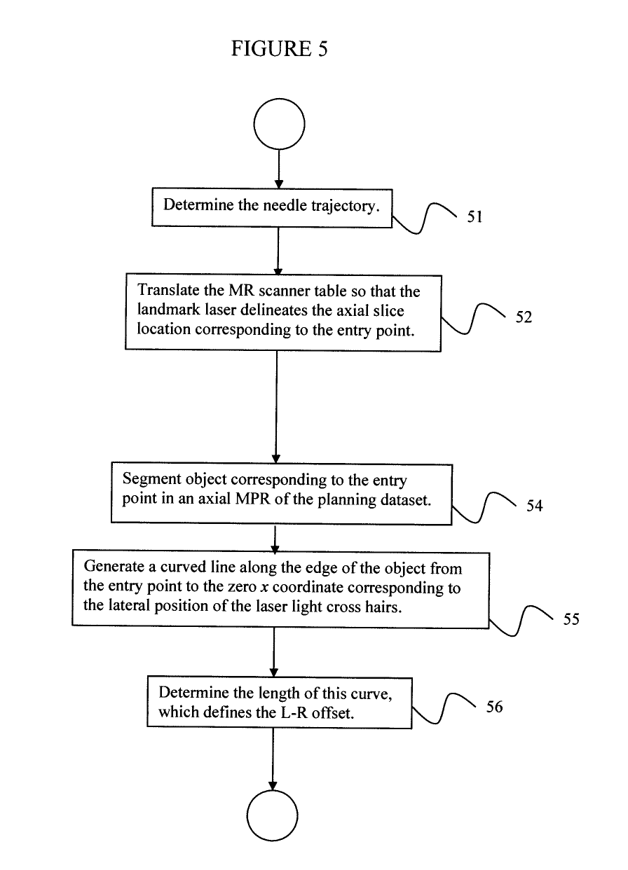

FIG. 5 is a flowchart of a method according to an embodiment of the invention for localizing a skin entry site. A first step 51 of a percutaneous needle intervention according to an embodiment of the invention is to plan the needle trajectory. A trajectory can be defined using a planning software application with a highly resolved 3D dataset, and the planned trajectory can be used to localize the prescribed entry point (eₓ, e_{y}, e_{z}) on the patient's skin without further imaging using the built-in landmark laser that is part of any MR scanner, and image processing methods. FIG. 1 illustrates the use of a built-in landmark laser for physical entry point localization, and shows the landmark laser 11 and the laser cross hairs 12 on the patient's skin. The position defined on the patient by the landmark laser, when moved into the magnet, will coincide with the isocenter of the magnet and define the origin of the patient coordinate system used by the DICOM standard. FIG. 2 illustrates how a planning application defines the trajectory by setting entry and target points in MPR planes extracted from the 3D dataset. The upper right image of FIG. 2 is a volume rendering of the target area of the patient's body, and the other 3 images are MPR's along mutually orthogonal planes, and the cross hairs in each image indicates the plane of the other 2 images. The 3 planes are, clockwise from the lower right, left-right, axial, and head-foot.

An approach according to an embodiment of the invention for physically locating an entry site on a patient includes a superior-inferior localization step and a lateral localization step.

Referring again to FIG. 5, a superior-inferior localization step 52 according to an embodiment of the invention is performed by translating the MR scanner table so that the landmark laser delineates the axial slice location corresponding to the entry point. The table movement tₘₒᵥₑ can be calculated by

$${t}_{move} = {d}_{iso , laser} + {t}_{curr\_pos} + \{ \begin{matrix}{e}_{z} \\ - {e}_{z}\end{matrix}$$

depending on whether the patient is

$$registered \{ \begin{matrix}head \\ feet\end{matrix}$$

first, where d_{iso,laser} is the distance between the laser light of the MR scanner and the isocenter of the magnet, t_{curr}_{_{—}}ₚₒₛ is the current table position and e_{z} is the z-coordinate of the planned entry point. Two cases need to be distinguished as the spatial information encoded in the DICOM image header is based on the patient-centered coordinate system. Thus, the coordinate system changes with respect to the patient registration.

In a lateral localization according to an embodiment of the invention, having moved the table by tₘₒᵥₑ the landmark laser light is switched on and the L-R offset from the laser cross-hairs is measured using an MR-compatible measuring tape. The L-R offset d_{l,r} is defined by the distance along the patient's surface from the planned entry point to the point marked on the patient's skin by the laser crosshairs. According to an embodiment of the invention, the L-R offset can be calculated using several image processing steps.

First, at step 54, the object, such as a patient abdomen, in an axial MPR of the planning dataset corresponding to the entry point is segmented. An exemplary, non-limiting segmentation technique is the minimum error thresholding technique, which starts by calculating the axial MPR Iₐ(u, v) in which the entry point lies, based on the 3-D coordinates of the entry point. The background can be characterized as an area of low signal, i.e. air, corrupted by noise. An exemplary, non-limiting minimum error thresholding segmentation method for different sized background and foreground datasets is that of Kittler, et al., Pattern Recognition, Vol. 19, No. 1, pgs 41-47, 1986, the contents of which are herein incorporated by reference in their entirety. The background and subject are modeled by two overlapping normal distributions with grey values g in the range [0, N-1]. An exemplary, non-limiting value of N is 4096. The distribution of the grey levels in the image forms a histogram h(g) which gives an estimate of the probability density function p(g) of the mixture population comprising grey levels of object and background pixels. Each of the two components p(g|i) of the mixture may be assumed to be normally distributed with mean μᵢ, standard deviation σⁱ and a priori probability Pᵢ such that

$$\begin{matrix}p ( g ) = {\sum }_{i = 1}^{2} {P}_{i} p ( g \vert i ) \\ where & ( 1 ) \\ p ( g \vert i ) = \frac{1}{\sqrt{2 π} {σ}_{i}} exp ( - \frac{{( g - {μ}_{i} )}^{2}}{2 {σ}_{i}^{2}} ) . & ( 2 )\end{matrix}$$

For given p(g|i) and Pᵢ there exists a grey level τ for which grey levels g satisfy

P₁p(g|1)>P₂p(g|2), g≦τ,

P₁p(g|1)<P₂p(g|2), g>τ. (3)

τ is the minimum error threshold at which the image should be binarised. Taking the logarithm of both sides in EQ. (3), this condition can be re-expressed as

$$\frac{{( g - {μ}_{1} )}^{2}}{{σ}_{1}^{2}} + log {σ}_{1}^{2} - 2 log {P}_{1} < \frac{{( g - {μ}_{2} )}^{2}}{{σ}_{2}^{2}} + log {σ}_{2}^{2} - 2 log {P}_{2} , g \leq τ , \\ \frac{{( g - {μ}_{1} )}^{2}}{{σ}_{1}^{2}} + log {σ}^{2} - 2 log {P}_{1} > \frac{{( g - {μ}_{2} )}^{2}}{{σ}_{2}^{2}} + log {σ}_{2}^{2} - 2 log {P}_{2} , g > τ , \qquad ( 4 )$$

The minimum error threshold is determined by the threshold level τ. The grey level data can be thresholded at some arbitrary level T, and each of the two resulting pixel populations can be modeled by a normal density h(g|i,T) with parameters μᵢ(T), σᵢ(T) and a priori probability Pᵢ(T) given, respectively,

$$\begin{matrix}{μ}_{i} ( T ) = \frac{{\sum }_{g = a}^{b} gh ( g )}{{P}_{i} ( T )} , {σ}_{i}^{2} = \frac{{\sum }_{g = a}^{b} {( g - {μ}_{i} ( T ) )}^{2} h ( g )}{{P}_{i} ( T )} \\ and & ( 5 ) \\ {P}_{i} ( T ) = {\sum }_{g = a}^{b} h ( g ) \\ where \\ a = \{ \begin{matrix}0 , & i = 1 \\ T + 1 , & i = 2\end{matrix} and b = \{ \begin{matrix}T , & i - 1 \\ N - 1 , & i = 2.\end{matrix} & ( 6 )\end{matrix}$$

Now using the models h(g|i,T), i=1, 2, the conditional probability e(g, T) of grey level g being replaced in the image by a correct binary value is given by

$$e ( g , T ) = \frac{h ( g \vert i , T ) {P}_{i} ( T )}{h ( g )} , \qquad ( 7 )$$

where i=1 for g≦T and i=2 for g>T. As h(g) is independent of both i and T, the denominator in EQ. (7) may be ignored. Taking the logarithm of the numerator in EQ. (7) and multiplying the result by −2 yields

$$ɛ ( g , T ) = [ \frac{g - {μ}_{i} ( T )}{{σ}_{i} ( T )} ] + 2 log {σ}_{i} ( T ) - 2 log {P}_{i} ( T ) .$$

The average performance figure for the whole image can then be given by

$$J ( T ) = {\sum }_{g} h ( g ) \cdot ɛ ( g , T ) ,$$

The value of threshold T that minimizes the criterion J(ε,T) will give the best fit model and therefore the minimum error threshold.

J(T) can be expressed as

J(T)=1+2[P₁(T)log σ₁(T)+p₂(T)log σ₂(T)]−2[P₁(T)log P₁(T)+P₂(T)log P₂(T)].

The minimum error threshold Tₒₚₜ is thus given by arg_{T}min J(T) which can be computed in an iterative fashion as described in Kittler. The binary image B(u, v) is then calculated from

$$B ( u , v ) = \{ \begin{matrix}0 , & {I}_{a} ( u , v ) < {T}_{opt} , \\ 1 , & otherwise .\end{matrix}$$

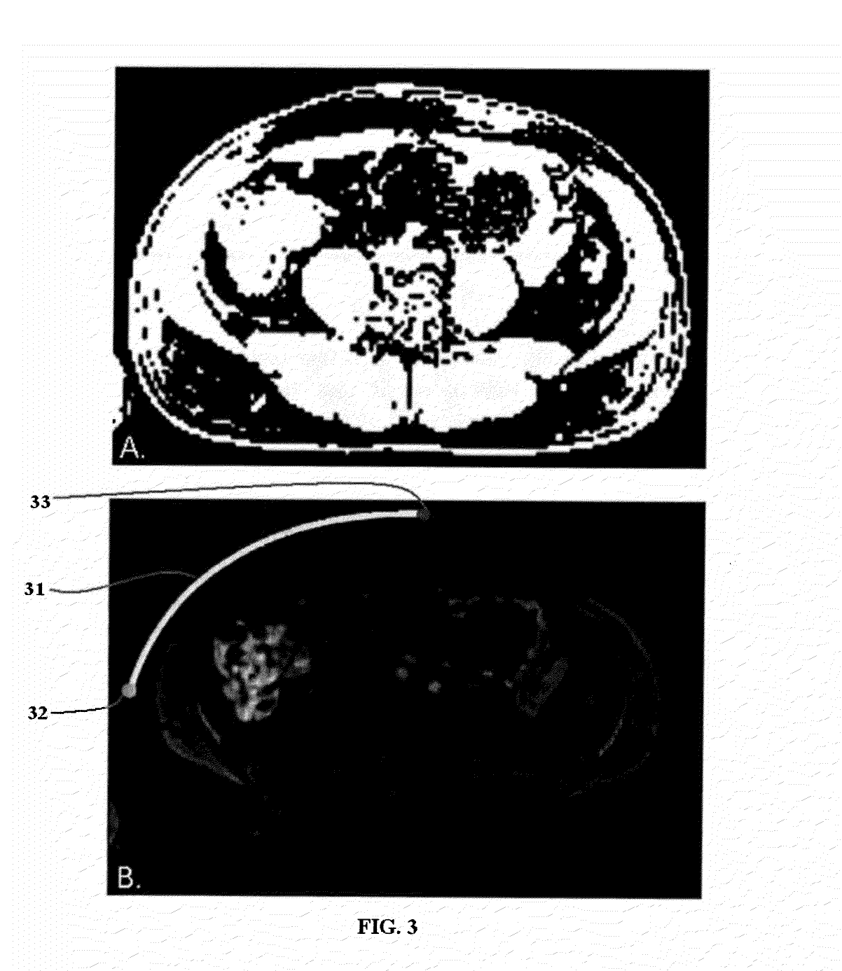

Second, at step 55, a curved line is generated along the edge of the thresholded object from the entry point to the zero x coordinate which corresponds to the lateral position of the laser light cross hairs. Finally, at step 56, the length of this curve is determined, which defines the L-R offset. Once the curve length has been determined, the MR compatible tape can be laid on the patient's skin, and the entry point can be physically located from the calculated curve length. FIGS. 3(A)-(B) depicts an axial slice corresponding to the skin entry site, used for calculating the L-R offset. FIG. 3(A) shows a binary threshold image used to identify the patient's skin, and FIG. 3(B) shows the calculated curve 31 from the planned entry point 32 to the zero x coordinate 33 defining the L-R offset.

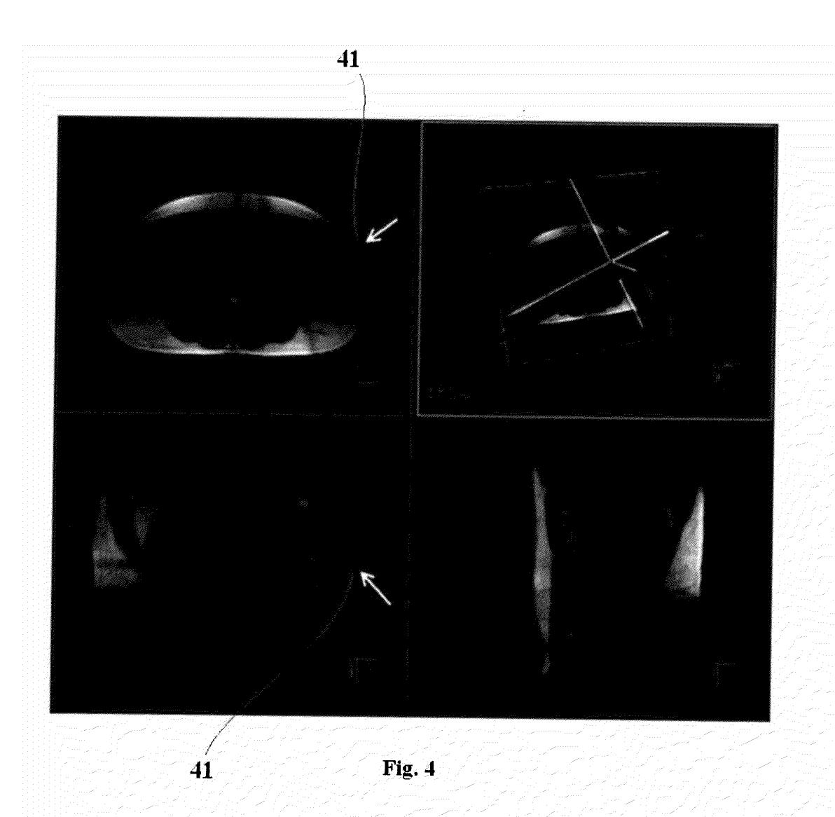

For validation of an entry point localization method according to an embodiment of the invention, a volunteer study was performed using a Siemens MAGNETOM Avanto 1.5T MR scanner. 20 entry sites were planned using a high resolution 3D dataset acquired under breath-hold conditions (VIBE: TR/TE 4.74/2.38 ms, flip-angle 10°, field-of-view 261×380 mm, matrix 110×160, slice thickness 2 mm). Each entry pint was localized on the volunteer's skin using an approach according to an embodiment of the invention, and a fish-oil capsule was placed on the identified site. For verification of the correct entry point localization, two imaging planes were prescribed along the planned trajectory orthogonal to each other using an automatic slice alignment approach. The fish-oil capsule was correctly placed if it could be seen in both slices. FIG. 4 illustrates verification imaging slices aligned along the planned trajectory that confirm the correct positioning of the fish-oil capsules 41 at the prescribed entry point, indicated by the arrows in the 2 left images. The upper right image of FIG. 4 is a volume rendering of the target area of the patient's body, and the other 3 images are MPR's along mutually orthogonal planes, which are, clockwise from the lower right, left-right, axial, and head-foot. Note the 3 mutually orthogonal planes are indicated in the volume rendering in the upper right.

The capsule was successfully placed at the planned entry point in 18 out of 20 cases. In the two unsuccessful cases, the capsule could be identified slightly off the planned path in the verification images. A possible explanation for the misplacement might be that the capsule moved between placement and imaging due to poor fixation.

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

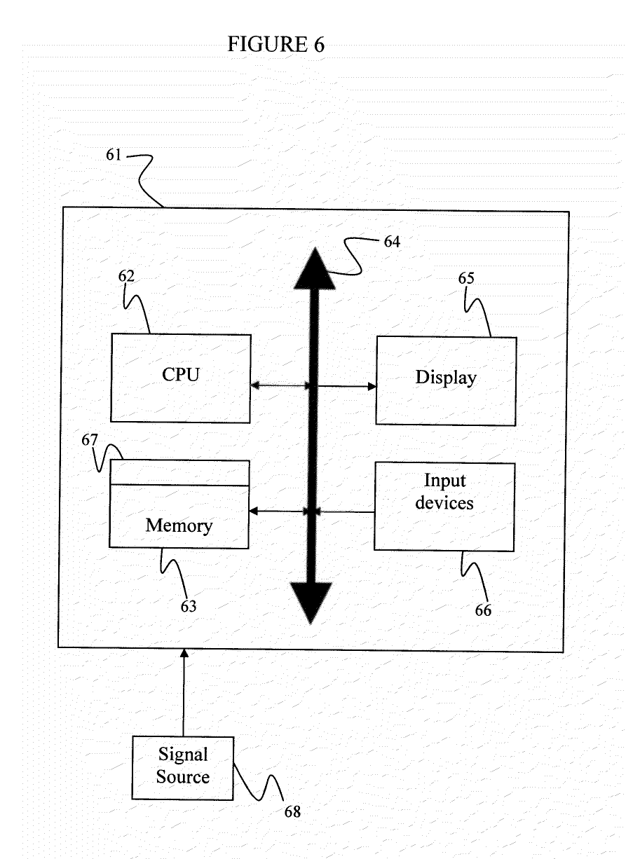

FIG. 6 is a block diagram of an exemplary computer system for implementing a method for localizing a skin entry site, according to an embodiment of the invention. Referring now to FIG. 6, a computer system 61 for implementing the present invention can comprise, inter alia, a central processing unit (CPU) 62, a memory 63 and an input/output (I/O) interface 64. The computer system 61 is generally coupled through the I/O interface 64 to a display 65 and various input devices 66 such as a mouse and a keyboard. The support circuits can include circuits such as cache, power supplies, clock circuits, and a communication bus. The memory 63 can include random access memory (RAM), read only memory (ROM), disk drive, tape drive, etc., or a combinations thereof. The present invention can be implemented as a routine 67 that is stored in memory 63 and executed by the CPU 62 to process the signal from the signal source 68. As such, the computer system 61 is a general purpose computer system that becomes a specific purpose computer system when executing the routine 67 of the present invention.

The computer system 61 also includes an operating system and micro instruction code. The various processes and functions described herein can either be part of the micro instruction code or part of the application program (or combination thereof) which is executed via the operating system. In addition, various other peripheral devices can be connected to the computer platform such as an additional data storage device and a printing device.

It is to be further understood that, because some of the constituent system components and method steps depicted in the accompanying figures can be implemented in software, the actual connections between the systems components (or the process steps) may differ depending upon the manner in which the present invention is programmed. Given the teachings of the present invention provided herein, one of ordinary skill in the related art will be able to contemplate these and similar implementations or configurations of the present invention.

While the present invention has been described in detail with reference to exemplary embodiments, those skilled in the art will appreciate that various modifications and substitutions can be made thereto without departing from the spirit and scope of the invention as set forth in the appended claims.

What is claimed is:

1. A method for localizing a skin entry point on a patient for a percutaneous intervention, comprising the steps of:
planning a needle trajectory for the percutaneous intervention using a 3D planning image dataset and a planning application;
performing a superior-inferior localization of an imaging scanner table containing an imaging scanner using the needle trajectory; and
performing a lateral localization of the skin entry point using the needle trajectory.

2. The method of claim 1 wherein performing a superior-inferior localization of an imaging scanner table comprises translating the imaging scanner table so that a landmark laser of the imaging scanner delineates an axial slice location in the 3D planning image dataset corresponding to the skin entry point.

3. The method of claim 2, wherein translating an imaging scanner table is determined from
tₘₒᵥₑ=d_{iso,laser}+t_{curr}_{_{—}}ₚₒₛ±e_{z}, wherein d_{iso,laser} is a distance between the landmark laser and an isocenter of the imaging scanner magnet, t_{curr}_{_{—}}ₚₒₛ is a current scanner table position and e_{z} is a z-coordinate of the skin entry point.

4. The method of claim 3, wherein the e_{z} term is added if the patient is registered head first with the imaging scanner, and the the e_{z} term is subtracted if the patient is registered feet first with the imaging scanner.

5. The method of claim 1, wherein performing a lateral localization of the skin entry point comprises:
segmenting an object in the axial slice corresponding to the skin entry point;
generating a curved line along an edge of the segmented object from the skin entry point to a zero x coordinate; and
determining a length of the curved line, wherein the curve length defines a lateral offset of the skin entry point.

6. The method of claim 5, wherein a landmark laser of the imaging scanner is illuminating the zero x coordinate with cross hairs, and the skin entry point corresponds to a lateral position of the laser light cross hairs.

7. The method of claim 5, wherein segmenting an object is performed using a minimum error thresholding technique.

8. A system for localizing a skin entry point on a patient for a percutaneous intervention, comprising:
an imaging scanner disposed on an imaging scanner table, said imaging scanner configured to acquire imaging data from a patient and including a landmark laser;
a planning application configured to plan a needle trajectory for the percutaneous intervention using a 3D planning image dataset;
a superior-inferior localizer configured to translate the imaging scanner table so that the landmark laser of the imaging scanner delineates an axial slice location in the 3D planning image dataset corresponding to the skin entry point; and
a lateral localizer configured to measure a distance along the patient's skin from the skin entry point to a point marked on the patient's skin by the landmark laser.

9. The system of claim 8, wherein the imaging scanner table translation is determined from tₘₒᵥₑ=d_{iso,laser}+t_{curr}_{_{—}}ₚₒₛ±e_{z}, wherein d_{iso,laser} is a distance between the landmark laser and an isocenter of the imaging scanner magnet, t_{curr}_{_{—}}ₚₒₛ is a current scanner table position, e_{z} is a z-coordinate of the skin entry point, wherein the e_{z} term is added if the patient is registered head first with the imaging scanner, and the the e_{z} term is subtracted if the patient is registered feet first with the imaging scanner.

10. The system of claim 8, wherein the imaging scanner is a magnetic resonance imaging scanner.

11. A non-transitory program storage device readable by a computer, tangibly embodying a program of instructions executed by the computer to perform the method steps for localizing a skin entry point on a patient for a percutaneous intervention, the method comprising the steps of:
planning a needle trajectory for the percutaneous intervention using a 3D planning image dataset and a planning application;
performing a superior-inferior localization of an imaging scanner table containing an imaging scanner using the needle trajectory; and
performing a lateral localization of the skin entry point using the needle trajectory.

12. The computer readable program storage device of claim 11, wherein performing a superior-inferior localization of an imaging scanner table comprises translating the imaging scanner table so that a landmark laser of the imaging scanner delineates an axial slice location in the 3D planning image dataset corresponding to the skin entry point.

13. The computer readable program storage device of claim 12, wherein translating an imaging scanner table is determined from
tₘₒᵥₑ=d_{iso,laser}+t_{curr}_{_{—}}ₚₒₛ±e_{z}, wherein d_{iso,laser} is a distance between the landmark laser and an isocenter of the imaging scanner magnet, t_{curr}_{_{—}}ₚₒₛ is a current scanner table position and e_{z} is a z-coordinate of the skin entry point.

14. The computer readable program storage device of claim 13, wherein the e_{z} term is added if the patient is registered head first with the imaging scanner, and the the e_{z} term is subtracted if the patient is registered feet first with the imaging scanner.

15. The computer readable program storage device of claim 11, wherein performing a lateral localization of the skin entry point comprises:
segmenting an object in the axial slice corresponding to the skin entry point;
generating a curved line along an edge of the segmented object from the skin entry point to a zero x coordinate; and
determining a length of the curved line, wherein the curve length defines a lateral offset of the skin entry point.

16. The computer readable program storage device of claim 15, wherein a landmark laser of the imaging scanner is illuminating the zero x coordinate with cross hairs, and the skin entry point corresponds to a lateral position of the laser light cross hairs.

17. The computer readable program storage device of claim 15, wherein segmenting an object is performed using a minimum error thresholding technique.
